(19) United States
(12) Patent Application Publication
(10) Pub. No.: US 2005/0276771 A1
(43) Pub. Date: Dec. 15, 2005

(54) Color-giving lip covering, color for lips, peelable cosmetics, and other cosmetics

(76) Inventors: Lewis Farsedakis, McLean, VA (US); Aryeh Elbaz, Vancouver (CA)

Correspondence Address: WHITHAM, CURTIS & CHRISTOFFERSON, P.C., 11491 SUNSET HILLS ROAD, SUITE 340, RESTON, VA, 20190, US

(21) Appl. No.: 11/149,152

(22) Filed: Jun. 10, 2005

Related U.S. Application Data
(60) Provisional application No. 60/578,456, filed on Jun. 10, 2004.

(51) Int. Cl. A61K 7/025

(52) U.S. Cl. 424/64

(57) ABSTRACT

Cosmetics with controllable removability are disclosed, such as a color-giving lip covering. Controlled removability of lip cosmetics, eye cosmetics and other cosmetics is advantageously provided by peeling when desired and only when desired.

### RELATED APPLICATION

This application claims benefit of U.S. provisional application No. 60/578,456 filed Jun. 10, 2004, titled “Color-giving lip covering, and color for lips.”

### FIELD OF THE INVENTION

This invention relates to cosmetics.

### BACKGROUND OF THE INVENTION

Many lipsticks, lip glosses and cosmetic lip products are known conventionally or have been disclosed in literature. E.g., U.S. Pat. No. 6,723,307 to Rose, III et al. (Bonne Bell, Inc.) dated Apr. 20, 2004 for “Cosmetic lip product with sour flavor;” U.S. Pat. No. 6,455,055 to Walling et al. (The Procter & Gamble Company) dated Sep. 24, 2002, for “Cosmetic compositions.” Lipsticks and other methods of coloring one's lips are widely used by many individuals.

Lip cosmetic products are one area of cosmetics generally. Many cosmetics are known and have been suggested. E.g., U.S. Pat. No. 6,596,285 to Kaneda et al. (Shiseido Co., Ltd.) for “Emulsified cosmetic face pack;” U.S. Pat. No. 6,298,864 to Gueret (L'Oreal) for “Device for applying a film-forming composition;” U.S. Patent Application No. 20030175328 dated Sep. 18, 2003 by Shefer et al., for “Patch for the controlled delivery of cosmetic, dermatological, and pharmaceutical active ingredients into the skin;” U.S. Patent Application No. 20030175333 dated Sep. 18, 2003 by Shefer et al., for “Invisible patch for the controlled delivery of cosmetic, dermatological, and pharmaceutical active ingredients onto the skin;” U.S. Pat. No. 6,589,541 to Halston et al. (Classified Cosmetics, Inc.), dated Jul. 8, 2003, for “Sprayable beautifying composition.”

U.S. Patent Application No. 2004/0143026 published Jul. 22, 2004, by Shah, is titled “Bioadhesive hydrophilic composition for treatment of mammalian skin.”

However, despite the demand for products for coloring lips and other skin and the work in the cosmetics area, conventional products and methods for lip-coloring and other skin-coloring still suffer from deficiencies, such as one or more of difficulty of controllable removal, problems with remaining in place as desired, unattractive appearance, cracking, etc.

### SUMMARY OF THE INVENTION

The present invention in an exemplary embodiment provides a method of coloring human skip (such as a human lip, eyelid, etc.) in which the material applied to the human skin (such as the lip, eyelid, etc.) is biocompatible and adheres to the skin where applied (such as the lip, eyelid, etc.) wherein the applied material includes a colourant. The applied material non-permanently and non-destructively adheres to the skin where applied (such as the lip, the eyelid, etc.), and is controllably removable from the skin where applied (such as the lip, etc.). In one embodiment, the applied material may be removed (e.g., peeled) as desired by a wearer wanting to remove the applied material but the applied material is not subject to being accidentally removed (such as, e.g., by kissing, eating, etc.) or dislodged. Most preferably, for making removal as desired easier, a pre-selected remover-substance or remover-solution or releasing agent may be applied.

In a preferred embodiment, the invention provides a color-giving lip covering comprising: a bioadherant component for adhering to a human lip and a colourant, with the color-giving lip covering having controllable removability (such as, e.g., removability controlled by applying a remover-solution (preferably a non-toxic remover-solution) to the lip covering to impart a peelable property to the lip covering; controllable removability via peeling as desired by a wearer of the applied lip covering. etc.), such as, e.g., lip coverings wherein before application to a lip, the lip covering is a liquid; lip coverings wherein upon application as a liquid to a lip, the liquid solidifies into a solid; lip coverings wherein peeling as desired may be accomplished after application of a remover solution; etc.

In another preferred embodiment, the invention provides an alcohol-free lip cosmetic, comprising: a bioadherant component for adhering to a human lip; and a colourant; wherein the lip cosmetic is alcohol-free, such as, e.g., alcohol-free lip cosmetics including an ingredient providing controllable removability.

The invention in a further preferred embodiment provides a peelable lip-coloring cosmetic, comprising: an emulsion that contains micro-, or nano-tubes, and/or microspheres suspended in the emulsion; a hydrophilized polymer; a film-forming composition (such as, e.g., a film-forming composition comprising one or more polymers and one or more solvents); diluted pigments; and solid microfibers (preferably natural solid microfibers which when dried create a mesh, such as, e.g., cotton microfibers), such as, e.g., peelable lip-coloring cosmetics including natural dyes; peelable lip-coloring cosmetics including preservatives, therapeutic agents, and waxes; and other peelable lip-coloring cosmetics.

### DESCRIPTION OF A PREFERRED EMBODIMENT

The present invention provides, in one preferred embodiment, a color-giving lip covering or a lip cosmetic comprising: a bioadherant component for adhering to a human lip; and a colourant. The color-giving lip cover has controllable removability, such as, e.g., the removability being controlled by applying a remover-solution (preferably a non-toxic remover-solution) to the lip covering to impart a peelable property to the lip covering; the removability being controlled via peeling as desired by a wearer of the applied lip covering (such as peeling as desired being accomplished after application of a remover solution, etc.); etc.

The bioadherant, the colourant and/or the ingredient providing controllable removability may be the same or different, i.e., are not required to be separate and different ingredients.

For the inventive lip coverings, before application to a lip, preferably the to-be-applied lip covering may be a liquid, a gel, a paste, etc. In such a case of a material (such as a liquid, a gel, a paste, etc.) being applied as a lip covering, preferably, upon application to a lip, the material being applied solidifies.

Examples of a bioadherant component for adhering to a human lip are bioadherant and/or bioadhesive components known in the art for use in liquid bandaging and liquid wound healing for applying to human skin. See, e.g., U.S. Pat. App. 200300143277 (National Starch and Chemical Co.); U.S. App. 20030232088 (Kimberly-Clark Worldwide); U.S. Pat. No. 6,683,120 (First Water Ltd); U.S. Pat. No. 6,803,420, to Cleary, for “Two-phase, water-absorbent bioadhesive composition;” U.S. Pat. No. 6,824,792 to Foreman, for “Bioadhesive composition.”

In a particularly preferred example, the present invention provides an alcohol-free lip cosmetic. Traditional lipsticks include a lot of alcohol to cause the product to stay on the lips; however, alcohol is very drying. The present invention, by using a bioadherant combined with a colourant, can provide an alcohol-free lip cosmetic.

In the most preferred embodiment of the invention, an inventive lip cosmetic includes minimal or no alcohol content. Optionally, inventive lip cosmetics may contain ingredients not expressly mentioned herein, such as flavourants, fillers, etc., so long as such an optional additional ingredient in the amount and manner in which it is used does not interfere with the functions described herein or with biocompatibility.

In another preferred embodiment, the present invention provides a temporary layer of artificial color for a part of human skin (such as, e.g., a lip, etc.) that is in contact with the actual skin of a wearer and remains in place until intentionally and controllably unpeeled. Preferably the temporary layer of artificial color is biocompatible.

The invention may be extended beyond lip cosmetics to cosmetics for other areas of human skin besides lips, such as, e.g., providing a controllably peelable skin cosmetic such as a cosmetic or concealer for an area of the face or other exterior skin surface; a controllably peelable eyeshadow; a controllably removable eyeliner; etc.

The invention may be further appreciated with regard to the following Example, which is by way of illustration and is not intended to limit the invention.

### EXAMPLE 1

In this Example, an inventive peelable cosmetic comprises the following components: an emulsion (such as, preferably, an emulsion that contains micro-, or nano-tubes, and/or microspheres (MNMSs)); a hydrophilized polymer; a film-forming composition (such as, preferably, a film-forming composition comprising one or more polymers and one or more solvents); diluted pigments (preferably including natural dyes); solid microfibers (preferably natural solid microfibers, such as cotton, which when dried create a mesh which helps the peelability of the formed film); and diluted pigments (and preferably natural dyes). The MNMSs are suspended in an emulsion, preferably with the help of emulsifying agents. The emulsion preferably contains preservatives, and therapeutic agents offering hydration or healing of dried lips, and waxes or other ingredients such as ingredients to provide “gloss” favored by consumers in the case of a lip cosmetic.

The hydrophilized polymer may be in the form, for example, of the outer shell of the MNMS being composed of a hydrophilized polymer. Hydrophilized polymers are well documented and are referred in the literature as pressure-sensitive adhesives (PSAs). The PSAs useable in this invention include, e.g., PSAs conventionally used in consumer and medical applications where a need exists for adhesion to a skin surface; PSAs characterized as being tacky and exhibiting instant tack when applied to a substrate; etc. Examples of polymers used to manufacture PSAs are, for example, acrylic and methacrylic esters homo- or copolymers, butyl rubber-based systems, silicones, urethanes, vinyl esters and amides, olefin copolymers materials, natural or synthetic rubbers, etc. PSAs generally are hydrophobic polymers, and to increase the bioadhesion of PSAs (which results in hydrophilicity), PSAs are “hydrophilized” by incorporation of non-tacky hydrophilic polymers and fillers into a hydrophilic adhesive.

In one preferred embodiment, the core of the MNMSs is comprised of a film-forming composition comprising one or more polymers and one or more solvents. The selection of solvents depends primarily on the type of polymers used. Suitable polymers, for example, include acrylic resin, soluble latex, EVA, polyurethane, and polyvinyl alcohol.

When the composition of this Example is applied using an applicator which can be a brush, or as a foam, the outer shell of the MNMSs provides the desired tackiness, whereas the rupture of the MNMSs through additional applied pressure releases the film forming agents. The emulsion is then covering the substrate with the coloring and therapeutic agents. The suspended fibers form the mesh upon drying which enhances the peelability.

It will be appreciated that variations and modifications from the embodiments set forth above may be made without departing from the spirit of the invention, and that such modifications are to be considered within the present invention.

1. A color-giving lip covering comprising:
a bioadherant component for adhering to a human lip and
a colourant, with the color-giving lip covering having controllable removability.

2. The lip covering of claim 1, wherein the removability is controlled by applying a remover-solution to the lip covering to impart a peelable property to the lip covering.

3. The lip covering of claim 1, wherein before application to a lip, the lip covering is a liquid.

4. The lip covering of claim 3, wherein upon application to a lip, the liquid solidifies into a solid.

5. The lip covering of claim 2, wherein the remover-solution is non-toxic.

6. The lip covering of claim 1, wherein the controllable removability is via peeling as desired by a wearer of the applied lip covering.

7. The lip covering of claim 6, wherein peeling as desired may be accomplished after application of a remover solution.

8. An alcohol-free lip cosmetic, comprising:
a bioadherant component for adhering to a human lip; and
a colourant; wherein the lip cosmetic is alcohol-free.

9. The alcohol-free lip cosmetic of claim 8, including an ingredient providing controllable removability.

10. A peelable lip-coloring cosmetic, comprising:
an emulsion that contains micro-, or nano-tubes, and/or microspheres suspended in the emulsion;
a hydrophilized polymer;
a film-forming composition;
diluted pigments; and
solid microfibers.

11. The peelable lip-coloring cosmetic of claim 10, wherein the film-forming composition comprises one or more polymers and one or more solvents.

12. The peelable lip-coloring cosmetic of claim 10, including natural dyes.

13. The peelable lip-coloring cosmetic of claim 10, including natural solid microfibers which when dried create a mesh.

14. The peelable lip-coloring cosmetic of claim 13, including cotton microfibers.

15. The peelable lip-coloring cosmetic of claim 10, including preservatives, therapeutic agents, and waxes.
